(19) United States
(12) Patent Application Publication
(10) Pub. No.: US 2021/0008383 A1
(43) Pub. Date: Jan. 14, 2021

(54) SHORT WAVE VISUALIZATION PROBE DEVICE

(71) Applicant: ZMI ELECTRONICS LTD., Kaohsiung City (TW)

(72) Inventors: Chia-Ho CHEN, Kaohsiung City (TW); Chen-Shen CHU, Kaohsiung City (TW); Li-Ming LIU, Kaohsiung City (TW)

(21) Appl. No.: 16/508,902

(22) Filed: Jul. 11, 2019

(51) Int. Cl. A61N 5/02

(52) U.S. Cl. CPC A61N 5/025

(57) ABSTRACT

A short wave visualization probe device adapted to be electrically connected to a short wave diathermy device and adapted to treat body tissues is provided. The visualization probe device includes a housing unit, an electrode unit and a visualization unit. The electrode unit is disposed in the housing unit, is adapted to be electrically connected to the diathermy device and to generate an electromagnetic field when being actuated by the diathermy device so as to perform short wave diathermy on the body tissues. The visualization unit is disposed on the housing unit and is configured to generate a visually perceptible effect in response to exposure to the electromagnetic field generated by the electrode unit.

### FIELD

The disclosure relates to a short wave probe device, more particularly to a short wave visualization probe device.

### BACKGROUND

Generally, a therapist or a patient determines the output power of probes of a conventional short wave diathermy device according to heat sensed thereby during diathermy treatment. However, for someone who suffers from a peripheral nervous system disorder or disease such as diabetes, brain injury, spinal cord injury, or multiple sclerosis, and thus cannot sense heat properly, he/she may not be able to determine whether the output power is suitable when treated by the short wave diathermy device. As a consequence, when the diathermy device is not working or the output power is affected by placement of electrodes of the diathermy device, efficiency of diathermy treatment may not be satisfied.

To detect the output power of the probes of a short wave diathermy device, a radio frequency power meter may be employed to measure electromagnetic wave generated by the diathermy device, or an inspection instrument cooperating with a tube lamp may be employed. However, these devices (radio frequency power meters or inspection instrument) are not readily available in a clinical setting. Additionally, a user may not know where on the diathermy device the detecting device (e.g., the inspection instruction) should approach to appropriately detect the output power of the diathermy device without affecting impedance matching of the entire circuit loop of the diathermy device and causing the output power to be unstable.

### SUMMARY

Therefore, an object of the disclosure is to provide a short wave visualization probe device capable of visualizing high frequency waves outputted by a diathermy device.

According to one aspect of the disclosure, a short wave visualization probe device is adapted to be electrically connected to a shortwave diathermy device and is adapted to treat body tissues. The short wave visualization probe device includes a housing unit, an electrode unit and a visualization unit. The housing unit is adapted to be disposed near the body tissues to be treated. The electrode unit is disposed in the housing unit, is adapted to be electrically connected to the short wave diathermy device and is configured to generate an electromagnetic field when being actuated by the short wave diathermy device so as to perform short wave diathermy on the body tissues. The visualization unit is disposed on the housing unit and is configured to generate a visually perceptible effect in response to exposure to the electromagnetic field generated by the electrode unit.

### BRIEF DESCRIPTION OF THE DRAWINGS

Other features and advantages of the disclosure will become apparent in the following detailed description of the embodiments with reference to the accompanying drawings, of which:

FIG. 1 is a schematic view of a short wave visualization probe device according to a first embodiment of the present disclosure being electrically connected to a short wave diathermy device for treating body tissues;

FIG. 2 is a schematic diagram of the short wave diathermy device being electrically connected to the short wave visualization probe device of the first embodiment;

FIG. 3 is a schematic side view of one of two wave visualizer of the short wave visualization probe device of the first embodiment;

FIG. 4 is a schematic view of the first embodiment, illustrating the wave visualizers emitting visible light in response to exposure to electromagnetic field when the first embodiment is actuated by the short wave diathermy device;

FIG. 5 is a schematic side view of one of the wave visualizers of a modification of the first embodiment including a tube lamp for emitting visible light; and

FIG. 6 is a schematic side view of one wave visualizer of the short wave visualization probe device according to a second embodiment of the present disclosure.

### DETAILED DESCRIPTION

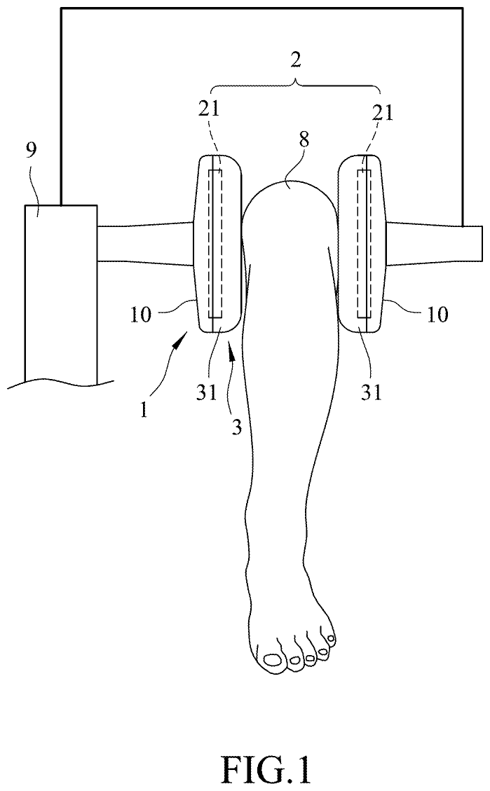

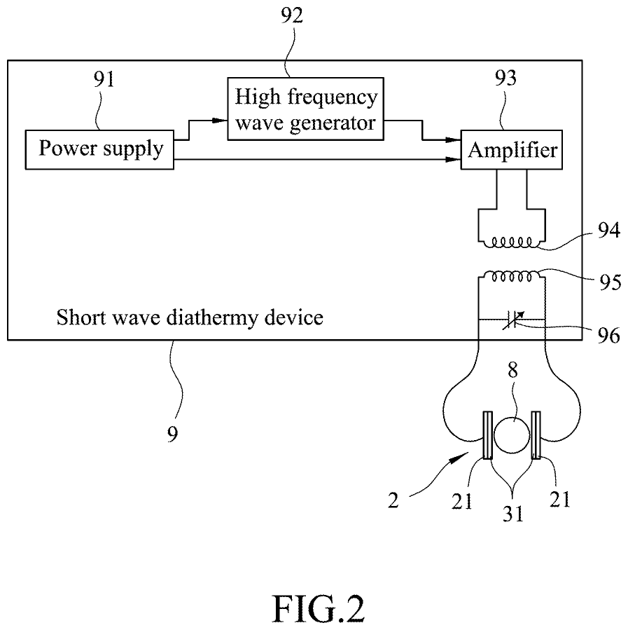

Referring to FIGS. 1 and 2, a short wave visualization probe device according to a first embodiment of this disclosure is adapted to be electrically connected to a short wave diathermy device 9 and is adapted to treat body tissues 8. The short wave visualization probe device includes a housing unit 1, an electrode unit 2 and a visualization unit 3.

The electrode unit 2 is disposed in the housing unit 1, is adapted to be electrically connected to the short wave diathermy device 9, and is configured to generate an electromagnetic field when being actuated by the short wave diathermy device 9 so as to perform short wave diathermy on the body tissues 8. The visualization unit 3 is disposed on the housing unit 1 and is configured to generate a visually perceptible effect in response to exposure to the electromagnetic field generated by the electrode unit 2.

In the first embodiment, the short wave diathermy device 9 includes a power supply 91 to be connected to the mains electricity, a high frequency wave generator 92, an amplifier 93, an oscillator coil 94, a resonator coil 95 and a variable capacitor 96. The variable capacitor 96 is connected in parallel to the resonator coil 95.

The housing unit 1 includes a pair of casings 10 spaced apart from each other and adapted to allow the body tissues 8 to be disposed therebetween. The electrode unit 2 includes a pair of electrodes 21 disposed respectively in the casings 10 and configured to provide high frequency waves of the electromagnetic field to be converted into heat by the body tissues 8. The high frequency waves of the electromagnetic field have a frequency ranging between 1 and 100 MHz, suitable for short wave diathermy. The electrodes 21 have opposite electric polarities, and are electrically and respectively connected to opposite terminals of the variable capacitor 96 (also the two opposite terminals of the resonator coil 95).

In this embodiment, the visualization unit 3 includes a pair of wave visualizers 31 disposed respectively on the casings 10 for generating the visually perceptible effect and indicating existence of the electromagnetic field generated by the electrodes 21. As shown in FIG. 1, the body tissues 8 to be treated are disposed between the electrodes 21 and between the wave visualizers 31, and cooperate with the electrodes 21 and the visualizers 31 to form a capacitor. The capacitor thus formed, the variable capacitor 96 and the resonator coil 95 can be referred to as a resonator or a “patient circuit”. The electrodes 21, when being actuated by the short wave diathermy device 9, provide the high frequency waves to perform short wave diathermy on the body tissues 8 and generate an electromagnetic field to cause the wave visualizers 31 to generate the visually perceptible effect in response to exposure to the electromagnetic field.

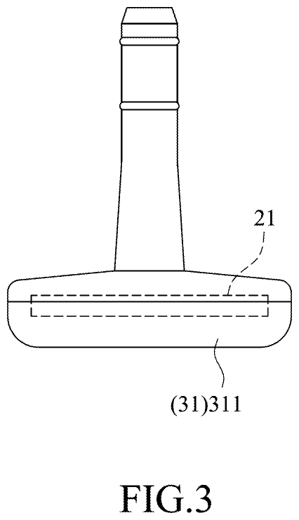

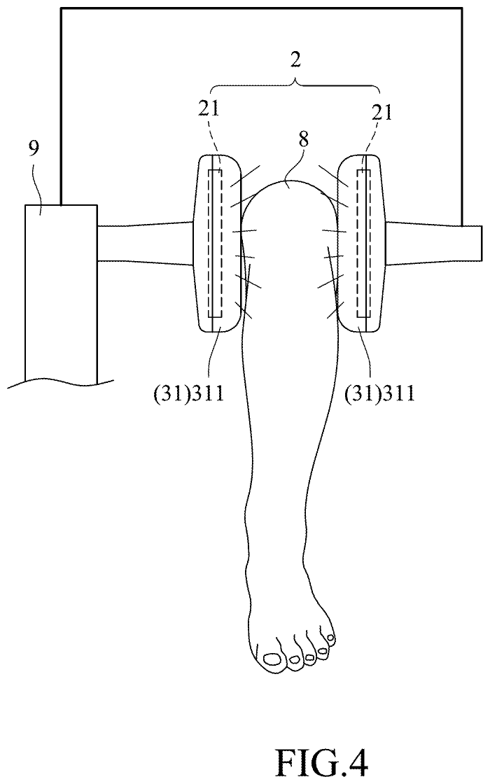

Further referring to FIGS. 3 and 4, each of the wave visualizers 31 is configured as a light guide cover 311 coated with a first electroluminescent material. In response to exposure to the electromagnetic field generated by the electrodes 21, the first electroluminescent material emits visible light to serve as the visually perceptible effect. The first electroluminescent material is, for example, powdered zinc sulfide doped with copper or silver, but the present disclosure is not limited to the disclosure herein. In other embodiments, the first electroluminescent material may be implemented by a polymer dispersed liquid crystal (PDLC) thin film, or an electrochromism material, such as viologens or phthalocyanine. In this way, a user of the short wave visualization probe device can tell the existence of the electromagnetic field generated by the electrodes 21 and tell the output power (as in power level) of the short wave diathermy device 9 at the electrodes 21 according to the visible light during treatment of the short wave diathermy device 9.

Additionally, when the electrode unit 2 is actuated by the short wave diathermy device 9 but the energy of high frequency waves provided by the electrodes 21 is not sufficient to be converted into heat by the body tissues 8 to be treated, the wave visualizers 31 are still able to emit light in response to exposure to the electromagnetic field generated by the electrodes 21, thereby providing a placebo effect to the user.

In this embodiment, the wave visualizers 31 are further coated with a second electroluminescent material that is different from the first electroluminescent material and that emits infrared light in response to exposure to the electromagnetic field generated by the electrodes 21. For example, the second electroluminescent material is gallium arsenide (GaAs) but the present disclosure is not limited to this example. Therefore, the short wave visualization probe device is capable of providing both short wave diathermy and infrared radiation treatment to the user at the same time.

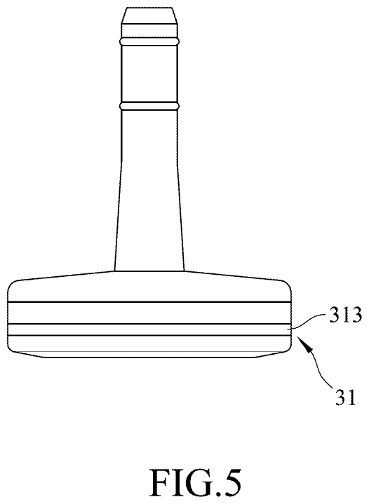

Referring to FIG. 5, in a modification of the first embodiment, each of the wave visualizers 31 (only one is depicted) includes a tube lamp 313 coated with the first and second electroluminescent materials to emit the visible light and infrared light in response to exposure to the electromagnetic field. That is to say, the modification of the first embodiment is also capable of providing short wave diathermy and infrared radiation treatment to the user at the same time.

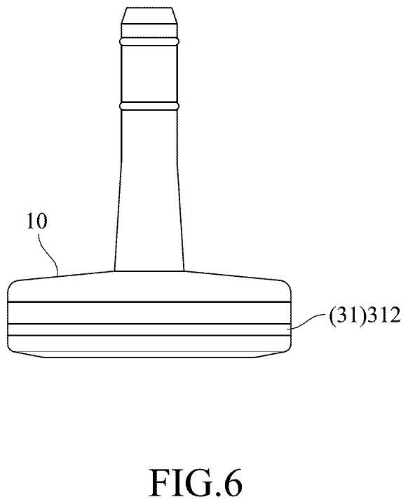

Referring to FIG. 6, part of the short wave visualization probe device according to a second embodiment of the present disclosure is shown. In this embodiment, each of the wave visualizers 31 (only one is depicted) is a light guide loop 312 disposed at a periphery of the respective one of the casings 10 (only one is depicted).

Similar to the first embodiment, the light guide loop 312 of each wave visualize 31 is coated with the first and second electroluminescent materials to emit visible light and infrared light in response to exposure to the electromagnetic field generated by the electrodes 21 (see FIG. 2).

When the electrode unit 2 (see FIG. 2) is actuated by the diathermy device 9, the body tissues 8 (see FIG. 2) disposed between the casings 10 convert high frequency waves provided by the electrodes 21 into heat so as to perform short wave diathermy, and the light guide loops 312 emit visible light and infrared light in response to exposure to the electromagnetic field generated by the electrodes 21.

To sum up, the first and second electroluminescent materials coated on the light guide covers 311, the tube lamps 313 or the light guide loops 312 are part of the patient circuit, thereby facilitating impedance matching of the entire circuit loop constituted by the short wave diathermy device 9, the short wave visualization probe device, and the body tissues 8 of the user. In addition, since the light guide covers 311, the tube lamps 313 or the light guide loops 312 are coated with the first and second electroluminescent materials, visualization or a visually perceptible effect is realized for the invisible high frequency waves of the electromagnetic field.

Further, when the energy of the high frequency waves or the output power of the short wave diathermy device 9 is relatively low, the wave visualizers 31 of the present disclosure would still emit light, thereby providing the placebo effect to the user. Finally, the short wave visualization probe device is capable of providing short wave diathermy and infrared radiation treatment to the user at the same time.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A short wave visualization probe device adapted to be electrically connected to a short wave diathermy device and adapted to treat body tissues, the short wave visualization probe device comprising:
a housing unit adapted to be disposed near the body tissues to be treated;
an electrode unit disposed in said housing unit, adapted to be electrically connected to the short wave diathermy device and configured to generate an electromagnetic field when being actuated by the short wave diathermy device so as to perform short wave diathermy on the body tissues; and
a visualization unit disposed on said housing unit and configured to generate a visually perceptible effect in response to exposure to the electromagnetic field generated by said electrode unit.

2. The short wave visualization probe device as claimed in claim 1, wherein said housing unit includes a pair of casings spaced apart from each other and adapted to allow the body tissues to be disposed therebetween, said electrode unit including a pair of electrodes disposed respectively in said casings and configured to provide high frequency waves of the electromagnetic field to be converted into heat by the body tissues, said visualization unit including a pair of wave visualizers disposed respectively on said casings for generating the visually perceptible effect and indicating existence of the electromagnetic field generated by said electrodes.

3. The short wave visualization probe device as claimed in claim 2, wherein each of said wave visualizers of said visualization unit is configured to generate visible light in response to exposure to the electromagnetic field generated by said electrodes to serve as the visually perceptible effect.

4. The short wave visualization probe device as claimed in claim 3, wherein each of said wave visualizers is coated with a first electroluminescent material that emits the visible light in response to exposure to the electromagnetic field generated by said electrodes.

5. The short wave visualization probe device as claimed in claim 4, wherein each of said wave visualizers is further coated with a second electroluminescent material that is different from the first electroluminescent material and that emits infrared light in response to exposure to the electromagnetic field generated by said electrodes.

6. The short wave visualization probe device as claimed in claim 5, wherein each of said wave visualizers is a light guide cover.

7. The short wave visualization probe device as claimed in claim 5, wherein each of said wave visualizers is a light guide loop disposed at a periphery of the respective one of said casings.

8. The short wave visualization probe device as claimed in claim 3, wherein each of said wave visualizers includes a tube lamp configured to emit the visible light in response to exposure to the electromagnetic field generated by said electrodes to serve as the visually perceptible effect.

9. The short wave visualization probe device as claimed in claim 8, wherein said tube lamp of each of said wave visualizers is further configured to emit infrared light in response to exposure to the electromagnetic field generated by said electrodes.
